(19) United States
(12) Patent Application Publication
(10) Pub. No.: US 2015/0182240 A1
(43) Pub. Date: Jul. 2, 2015

(54) ULTRASONIC TONGUE SCRAPER

(71) Applicant: Goldspire Group Limited, Causeway Bay (HK)

(72) Inventors: Alex Anthony Wawiluk, Monarch Beach, CA (US); Nicolas Alex Wawiluk, Monarch Beach, CA (US)

(73) Assignee: Goldspire Group Limited, Causeway Bay (HK)

(21) Appl. No.: 14/409,418

(22) PCT Filed: Jun. 28, 2013
(86) PCT No.: PCT/US2013/048485

§ 371 Date: Dec. 18, 2014

Related U.S. Application Data
(60) Provisional application No. 61/666,201, filed on Jun. 29, 2012.

(51) Int. Cl. A61B 17/24; A61N 7/00

(52) U.S. Cl. CPC A61B 17/244; A61N 7/00; A61B 2017/00473

(57) ABSTRACT

Apparatuses and methods clean and/or scrape bacterial build-up and other undesirable material from the surface of the tongue via an ultrasonic tongue scraper. The ultrasonic tongue scraper can provide manual scraping as well as mechanical vibration and ultrasound to assist the manual scraping.

### CROSS-REFERENCE TO RELATED APPLICATIONS

This application claims the benefit of U.S. Provisional Application No. 61/666,201, filed Jun. 29, 2012, entitled ULTRASONIC TONGUE SCRAPER, the contents of which are hereby incorporated by reference in their entireties as if fully set forth herein. The benefit of priority to the foregoing applications is claimed under the appropriate legal basis including, without limitation, under 35 U.S.C. §119(e).

### BACKGROUND

1. Field

Embodiments relate to the field of dental care, and, in particular, to apparatuses and methods for cleaning bacteria, including bacterial biofilm build-up, and other undesirable material from the surface of the tongue.

2. Description of the Related Art

The surface of the tongue can be a host for various undesirable materials, including but not limited to bacteria, food debris, fungi, and dead cells. Such bacteria and other materials on the surface of the tongue can be the cause of bad breath, bleeding, and infections among others. With the development of new technologies, various tongue cleaners and/or tongue cleaning methods are available to remove such bacteria and other materials from the surface of the tongue. Most tongue cleaners provide simple manual scraping of bacteria and other materials to address this issue. In some cases, prior art tongue cleaners have utilized ultrasonic technology. Further details regarding the use of ultrasonic tongue cleaners are disclosed in PCT Patent Publication WO 2002/034145A1, which is hereby incorporated by reference. Additionally, U.S. Pat. No. 5,772,434 and U.S. Pat. No. 5,853,290 disclose ultrasonic tongue cleaners such as those used in dental chairs by dental professionals at a low ultrasonic frequency, around 24-28 kHz, such as those used in ultrasonic tooth scalers for the removal of tartar buildup. Both U.S. Pat. No. 5,772,434 and U.S. Pat. No. 5,853,290 are also hereby incorporated by reference. However, there is need to provide for improved apparatuses, methods, and systems for cleaning the tongue, especially in a non-professional environment, such as in the home.

### SUMMARY

Advancements in technology make it possible to effectively clean the surface of the tongue by removing bacteria and other undesirable materials. In some embodiments, an ultrasonic tongue scraper for removing bacteria, including bacterial biofilm, from a surface of a tongue comprises a body portion, wherein the body portion comprises a power source, a motor configured to generate mechanical vibration, and an ultrasound generator configured to generate ultrasound, and a tongue scraper head portion, wherein the tongue scraper head portion is selectively removable from the body portion, transmit mechanical and/or sonic vibration generated by the motor to assist the manual scraping, and/or transmit ultrasound generated by the ultrasound generator to further assist the manual scraping. In certain embodiments, the tongue scraper head portion comprises a leading edge that is shaped and configured to be used to contact the tongue and manually scrape bacteria, including bacterial biofilm, off of the surface of the tongue.

In certain embodiments, the transmitted ultrasound in the ultrasonic tongue scraper above assists the manual scraping by breaking up chains of bacteria on the surface of the tongue. In certain embodiments, the ultrasound generator in the ultrasonic tongue scraper above is configured to generate continuous or pulsating ultrasound waves at a frequency of about 1.6 MHz. In certain embodiments, the ultrasonic tongue scraper above further comprises a vibration frequency selection input configured to change a frequency of the mechanical vibration. In certain embodiments, the frequency of the mechanical vibration of the ultrasonic tongue scraper above is among one of about 18,000, about 9,000, and about 0 movements per minute. In certain embodiments, the ultrasonic tongue scraper above further comprises an ultrasound indicator configured to indicate whether ultrasound is being generated by the ultrasound generator. In certain embodiments, the tongue scraper head portion of the ultrasonic tongue scraper above is configured to be selectively replaced with a brush head portion.

In some embodiments, a method of scraping bacteria, including bacterial biofilm, from a surface of a tongue comprises attaching a tongue scraper head portion to a body portion, wherein the tongue scraper head portion is selectively removable from the body portion, manually scraping bacteria, including bacterial biofilm, off of the surface of the tongue by contacting the surface of the tongue with a leading edge of the tongue scraper head portion, applying mechanical vibration to assist the manual scraping, wherein the mechanical vibration is generated by the body portion, and applying ultrasound to assist the manual scraping, wherein the ultrasound is generated by the body portion. In further embodiments, no mechanical vibration is applied.

In certain embodiments, the method of scraping bacteria from a surface of a tongue above further comprises breaking up chains of bacteria on the surface of the tongue by applying ultrasound. In certain embodiments, the generated ultrasound in the method of scraping bacteria from a surface of a tongue above comprises a continuous or pulsating frequency of about 1.6 MHz. In certain embodiments, the method of scraping bacteria from a surface of a tongue above further comprises selecting a frequency of the mechanical vibration. In certain embodiments, the frequency of the mechanical vibration in the method of scraping bacteria from a surface of a tongue above is selected among one of about 18,000, about 9,000, and about 0 movements per minute. In certain embodiments, the ultrasonic tongue scraper in the method of scraping bacteria from a surface of a tongue above indicates whether ultrasound is being generated. In certain embodiments, the method of scraping bacteria from a surface of a tongue above further comprises detaching the tongue scraper head portion and attaching a brush head portion. In some embodiments, the head portion is not detachable.

For purposes of this summary, certain aspects, advantages, and novel features are described herein. It is to be understood that not necessarily all such advantages may be achieved in accordance with any particular embodiment. Thus, for example, those skilled in the art will recognize that the invention may be embodied or carried out in a manner that achieves one advantage or group of advantages as taught herein without necessarily achieving other advantages as may be taught or suggested herein.

### BRIEF DESCRIPTION OF THE DRAWINGS

The foregoing and other features, aspects and advantages are described in detail below with reference to the drawings of various embodiments, which are intended to illustrate and not to limit the disclosure. The drawings comprise the following figures in which:

FIGS. 1A-1B depict examples of embodiments of ultrasonic tongue scraper head portions of ultrasonic tongue scrapers.

FIG. 2 depicts an example of one embodiment of a body portion of an ultrasonic tongue scraper.

FIG. 3 depicts an example of one embodiment of a replaceable brush head portion.

### DETAILED DESCRIPTION OF THE EMBODIMENTS

Embodiments of the invention will now be described with reference to the accompanying figures. The terminology used in the description presented herein is not intended to be interpreted in any limited or restrictive manner, simply because it is being utilized in conjunction with a detailed description of certain specific embodiments of the invention. Furthermore, embodiments of the invention may comprise several novel features, no single one of which is solely responsible for its desirable attributes or which is essential to practicing the inventions herein described.

The surface of the tongue can be a host for various undesirable materials, including but not limited to bacteria, food debris, fungi, and dead cells. Over 800 types of bacteria are known to be found in the average mouth. Certain portions of the tongue, such as the posterior dorsum of the tongue for example, can provide ideal habitats for anaerobic bacteria, where they are relatively undisturbed by normal activity and flourish under a coating of food debris. When left on the tongue, such bacteria can produce foul smells of indole, skatole, polyamines, and/or volatile sulfur compounds among others. In fact, the tongue is known to be the most common source of mouth related halitosis or bad breadth.

Cleaning of the tongue surface is known to be an effective solution to remove such bacteria for preventing bad breath and for general hygienic and health reasons. Tongue cleaners and/or scrapers are generally employed for such purposes and are used to manually scrape off bacterial biofilm, debris, and mucus present on the surface of the tongue. As used herein, the terms “tongue cleaner(s)” and “tongue scraper(s)” may be used interchangeably.

However, tongue cleaners that rely only on manual scraping have inherent limitations. For example, manual tongue cleaners will only clean portions of a tongue surface where there has been sufficient contact and force thereof between the manual tongue cleaner and the surface of the tongue. In other words, if the area and/or force of contact between a manual tongue cleaner and a portion of the tongue surface is insufficient, bacteria and/or other materials present on that portion of the tongue surface will not be removed.

Embodiments described herein provide improved apparatuses and methods of removing bacteria and/or other materials from the surface of the tongue. More particularly, ultrasonic tongue scrapers described in connection with embodiments described herein not only provide manual scraping but also generate mechanical vibration and ultrasound to further aid and assist manual scraping.

Overview of Structure

In an embodiment, an ultrasonic tongue scraper comprises a body portion 200 and a tongue scraper head portion 100. In some embodiments, the tongue scraper head portion 100 is selectively removable from the body portion 200. For example, an elongated end 215 of the body portion is configured to be placed inside the tongue scraper head portion 100 in certain embodiments to engage the tongue scraper head portion 100. In some embodiments, the tongue scraper head portion 100 can be selectively replaced with a brush head portion 300 that is configured to be used in connection with brushing teeth. In other embodiments, the tongue scraper head portion 100 is permanently or semi-permanently connected to the body portion 200 and cannot be selectively removed from the body portion 200.

Tongue Scraper Head Portion

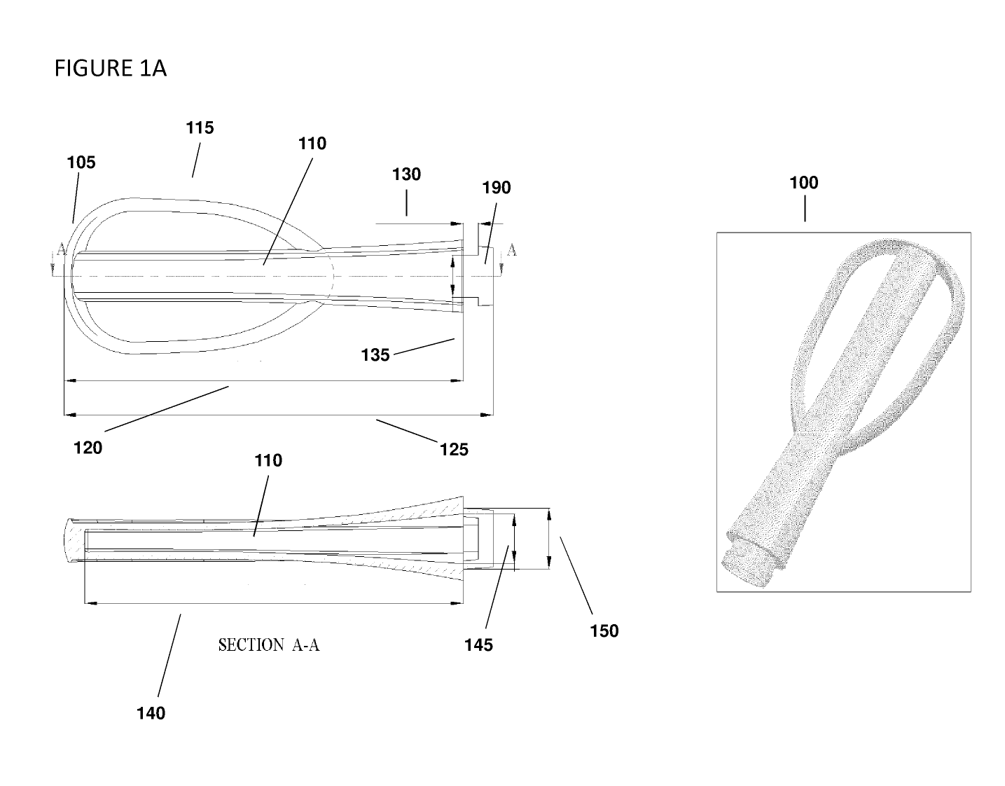

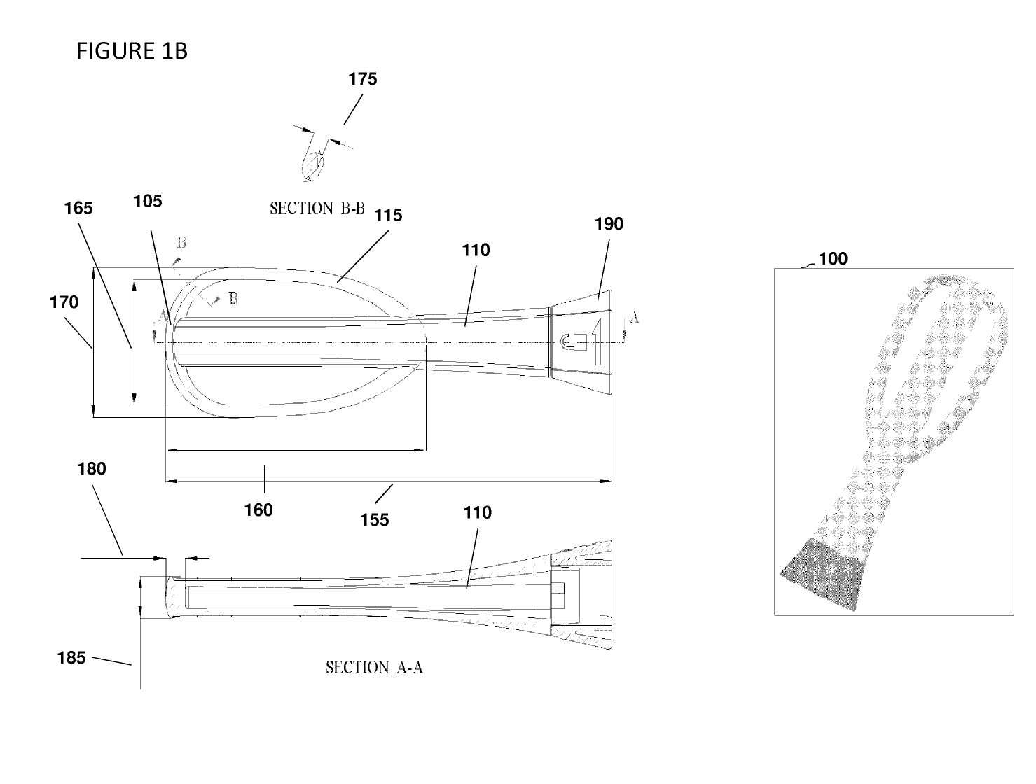

FIGS. 1A-1B illustrate examples of embodiments of ultrasonic tongue scraper head portions 100 of ultrasonic tongue scrapers. In an embodiment, the tongue scraper head portion 100 is configured to come into contact with the surface of a tongue to scrape off bacteria and/or other materials. In some embodiments, the tongue scraper head portion 100 is substantially circular or oval-shaped. In certain embodiments, the tongue scraper head portion 100 is substantially square or triangular in shape with or without rounded corners. In some embodiments, the tongue scraper head portion 100 is substantially open or anchor shaped.

In certain embodiments, distance D1 120 is representative of the distance of a section of the head portion. For example, this distance can be at least about 5 mm, at least about 10 mm, at least about 15 mm, least about 20 mm, at least about 25 mm, at least about 30 mm, at least about 35 mm, at least about 40 mm, at least about 45 mm, at least about 50 mm, at least about 55 mm, at least about 60 mm, at least about 65 mm, at least about 70 mm, at least about 75 mm, at least about 80 mm, at least about 85 mm, at least about 90 mm, at least about 95 mm, at least about 100 mm, at least about 150 mm, or more than 150 mm. In some embodiments, distance D2 125 is representative of a length of another section of the head portion. For example, this distance may be at least about 5 mm, at least about 10 mm, at least about 15 mm, least about 20 mm, at least about 25 mm, at least about 30 mm, at least about 35 mm, at least about 40 mm, at least about 45 mm, at least about 50 mm, at least about 55 mm, at least about 60 mm, at least about 65 mm, at least about 70 mm, at least about 75 mm, at least about 80 mm, at least about 85 mm, at least about 90 mm, at least about 95 mm, at least about 100 mm, or more than 100 mm.

In some embodiments, distance D3 130 is representative of the distance of a section of the head portion 100. For example, the distance can be less than 0.5 mm, at least about 0.5 mm, at least about 1.0 mm, at least about 1.5 mm, at least about 2.0 mm, at least about 2.5 mm, at least about 3.0 mm, at least about 3.5 mm, at least about 4.0 mm, or more than 4.0 mm. In certain embodiments, distance D4 135 is representative of the distance of a section of the head portion. For example, this distance may be less than 4.0 mm, at least about 4.0 mm, at least about 4.5 mm, at least about 5.0 mm, at least about 6.0 mm, at least about 6.5 mm, at least about 7.0 mm, at least about 7.5 mm, at least about 8.0 mm, at least about 8.5 mm, or more than 9.0 mm.

Distance D5 140 can be representative of the distance of a portion of the stem 110 portion. For example, this distance can be less than about 5 mm, at least about 10 mm, at least about 15 mm, least about 20 mm, at least about 25 mm, at least about 30 mm, at least about 35 mm, at least about 40 mm, at least about 45 mm, at least about 50 mm, at least about 55 mm, at least about 60 mm, at least about 65 mm, at least about 70 mm, at least about 75 mm, at least about 80 mm, at least about 85 mm, at least about 90 mm, at least about 95 mm, at least about 100 mm, at least about 150 mm, or more than 150 mm.

In certain embodiments, distance D6 145 can be representative of the distance of a section of the head portion. For example, this distance can be less than about 5.0 mm, at least about 5.0 mm, at least about 5.5 mm, at least about 6.0 mm, at least about 6.5 mm at least about 7.0 mm, at least about 7.5 mm, at least about 8.0 mm, at least about 8.5 mm, at least about 9.0 mm, at least about 9.5 mm, at least about 10.0 mm, at least about 10.5 mm, at least about 11.0 mm, or more than 11.0 mm. Distance D7 150 can be representative of the distance of a section of the head portion. For example, this distance can be less than about 5.0 mm, at least about 5.0 mm, at least about 5.5 mm, at least about 6.0 mm, at least about 6.5 mm at least about 7.0 mm, at least about 7.5 mm, at least about 8.0 mm, at least about 8.5 mm, at least about 9.0 mm, at least about 9.5 mm, at least about 10.0 mm, at least about 10.5 mm, at least about 11.0 mm, or more than 11.0 mm.

In one embodiment, D1 is about 63.4 mm, D2 is about 68.2 mm, D3 is about 2.4 mm, D4 is about 6.8 mm, D5 is about 60.8 mm, D6 is about 8.0 mm, and D7 is about 9.8 mm.

In some embodiments, distance D8 155 can be representative of the distance of the entire head portion including the locking portion 190. For example, this distance can be at least about 5 mm, at least about 10 mm, at least about 15 mm, least about 20 mm, at least about 25 mm, at least about 30 mm, at least about 35 mm, at least about 40 mm, at least about 45 mm, at least about 50 mm, at least about 55 mm, at least about 60 mm, at least about 65 mm, at least about 70 mm, at least about 75 mm, at least about 80 mm, at least about 85 mm, at least about 90 mm, at least about 95 mm, at least about 100 mm, at least about 150 mm, or more than 150 mm.

In some embodiments, the tongue scraper head portion 100 comprises a stem portion 110 and an outer portion 115. The stem portion 110 can be configured to be placed over an elongated end 215 of the body portion 200 to engage the tongue scraper head portion 100 with the body portion 200 in certain embodiments. In certain embodiments, the leading edge 105 of the outer portion is thicker than other parts of the outer portion 115 to allow bacteria and/or other material that is scraped by the leading edge 105 to accumulate.

In certain embodiments, distance D9 160 can be representative of the distance of a portion of the outer portion 115. For example, this distance can be at least about 5 mm, at least about 10 mm, at least about 15 mm, least about 20 mm, at least about 25 mm, at least about 30 mm, at least about 35 mm, at least about 40 mm, at least about 45 mm, at least about 50 mm, at least about 55 mm, at least about 60 mm, at least about 65 mm, at least about 70 mm, at least about 75 mm, at least about 80 mm, or more than 80 mm.

In some embodiments, distance D10 165 can be representative of the distance of the inner section of the outer portion of the head portion. For example, this distance can be less than 5 mm, at least about 5 mm, at least about 10 mm, at least about 15 mm, least about 20 mm, at least about 25 mm, at least about 30 mm, at least about 35 mm, at least about 40 mm, at least about 45 mm, or more than 45 mm. In certain embodiments, distance D11 170, can be representative of the distance of outer section of the outer portion of the head portion. For example, this distance can be less than 5 mm, at least about 5 mm, at least about 10 mm, at least about 15 mm, least about 20 mm, at least about 25 mm, at least about 30 mm, at least about 35 mm, at least about 40 mm, at least about 45 mm, or more than 45 mm.

In certain embodiments, distance D12 175 can be representative of the distance of a section of the outer portion 115. For example, the distance can be less than 0.5 mm, at least about 0.5 mm, at least about 1.0 mm, at least about 1.5 mm, at least about 2.0 mm, at least about 2.5 mm, at least about 3.0 mm, at least about 3.5 mm, at least about 4.0 mm, or more than 4.0 mm. In some embodiments, distance D13 180 can be representative of the distance of a section of the head portion 100. For example, the distance can be less than 0.5 mm, at least about 0.5 mm, at least about 1.0 mm, at least about 1.5 mm, at least about 2.0 mm, at least about 2.5 mm, at least about 3.0 mm, at least about 3.5 mm, at least about 4.0 mm, or more than 4.0 mm. In certain embodiments, distance D14 185 can be representative of the distance of a section of the head portion 100. For example, this distance can be less than about 5.0 mm, at least about 5.0 mm, at least about 5.5 mm, at least about 6.0 mm, at least about 6.5 mm at least about 7.0 mm, at least about 7.5 mm, at least about 8.0 mm, at least about 8.5 mm, at least about 9.0 mm, at least about 9.5 mm, at least about 10.0 mm, at least about 10.5 mm, at least about 11.0 mm, or more than 11.0 mm.

In one embodiment, D8 is about 73.6 mm, D9 is about 41.0 mm, D10 is about 21.0 mm, D11 is about 25.0 mm, D12 is about 2.3 mm, D13 is about 2.0 mm, and D14 is about 8.0 mm.

In certain embodiments, the stem portion 110 is configured to act as a pressure restrictor, thereby preventing a user from exerting an uncontrollable amount of pressure on the tongue. By preventing the user from exerting an uncontrollable amount of pressure on the tongue, the stem portion may act to prevent injury to the user. In some embodiments, the stem portion may be smooth. In certain embodiments, the outer portion 115 extends above the height of the stem 110, when the device is held flat with the majority of outer portion horizontal to the stem portion. In other embodiments, instead the stem portion extends beyond the height of the outer portion 115.

In certain embodiments, when the head portion 100 is held flat with the majority of the outer portion held horizontal to the stem portion, the edge of the outer portion can extend above the stem portion. For example, the edge could extend above the stem portion 110 by 0.5 mm or by 1.0 mm. In some embodiments the edge extends above the stem portion 110 by at least about 0.1 mm, at least about 0.2 mm, at least about 0.3 mm, at least about 0.4 mm, at least about 0.5 mm, at least about 0.6 mm, at least about 0.7 mm, at least about 0.8 mm, at least about 0.9 mm, at least about 1.0 mm, at least about 1.1 mm, at least about 1.2 mm, at least about 1.3 mm, at least about 1.4 mm, at least about 1.5 mm, or more than 1.5 mm.

In some embodiments, the tongue scraper head portion 100 has a locking mechanism 190 to keep the tongue scraper head portion 100 attached to the body portion 200. For example, the locking mechanism 190 can be a mechanical lock comprising a groove configured to be engaged when the lock is turned in a particular direction. When the lock is turned in an opposite direction, the mechanical lock can be unlocked in certain embodiments, allowing the tongue scraper head portion 100 to be removed from the body portion 200.

Body Portion

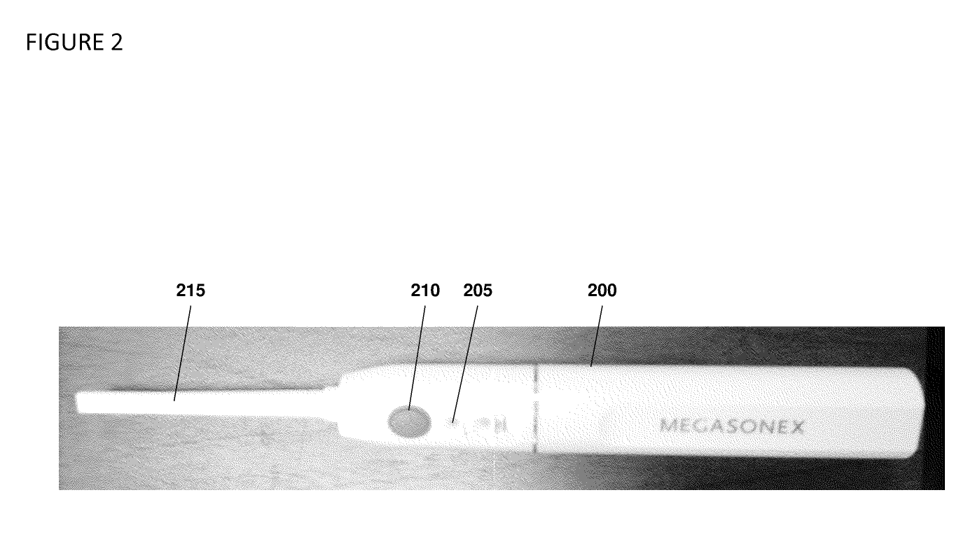

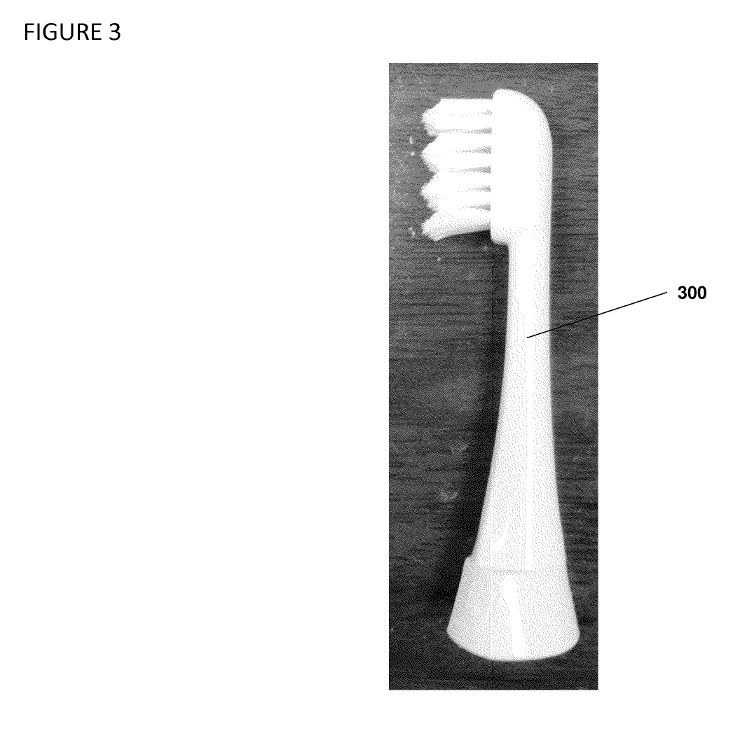

FIG. 2 illustrates an example of one embodiment of a body portion 200 of an ultrasonic tongue scraper. In an embodiment, the body portion 200 of an ultrasonic tongue scraper comprises a power source. The power source can be a battery, rechargeable or not, and/or any other power source that is currently known or to be developed. Further, in an embodiment, the body portion of an ultrasonic tongue scraper comprises a power button 210 or other input configured to turn the ultrasonic tongue scraper on and/or off.

In some embodiments, the body portion 200 of an ultrasonic tongue scraper comprises a motor and/or other means of vibration, such as, but not limited to, magnets or an audio generator for generating mechanical and/or sonic vibration. In some embodiments, the mechanical and/or sonic vibration is configured to be transmitted from the body portion 200 to and through the tongue scraper head portion 100. In certain embodiments, the mechanical and/or sonic vibration is transmitted to and through the tongue scraper head portion 100 via an elongated end 215 of the body portion 200 that is placed in the interior of the tongue scraper head portion 100. In some embodiments, the frequency of mechanical and/or sonic vibration can be controlled by a user via a vibration frequency selection button 210 or other input on the body portion 200. In other embodiments, the frequency of mechanical and/or sonic vibration cannot be controlled by a user.

In some embodiments, the motor is configured to vibrate at a particular frequency. For example, the motor can be configured to vibrate at a frequency of at least about 0 movements per minute, at least about 1,000 movements per minute, at least about 2,000 movements per minute, at least about 3,000 movements per minute, at least about 4,000 movements per minute, at least about 5,000 movements per minute, at least about 6,000 movements per minute, at least about 7,000 movements per minute, at least about 8,000 movements per minute, at least about 9,000 movements per minute, at least about 10,000 movements per minute, at least about 11,000 movements per minute, at least about 12,000 movements per minute, at least about 13,000 movements per minute, at least about 14,000 movements per minute, at least about 15,000 movements per minute, at least about 16,000 movements per minute, at least about 17,000 movements per minute, at least about 18,000 movements per minute, at least about 19,000 movements per minute, at least about 20,000 movements per minute, at least about 21,000 movements per minute, at least about 22,000 movements per minute, at least about 23,000 movements per minute, at least about 24,000 movements per minute, at least about 25,000 movements per minute, at least about 26,000 movements per minute, at least about 27,000 movements per minute, at least about 28,000 movements per minute, at least about 29,000 movements per minute, at least about 30,000 movements per minute, at least about 31,000 movements per minute, at least about 32,000 movements per minute, at least about 33,000 movements per minute, at least about 34,000 movements per minute, at least about 35,000 movements per minute, at least about 36,000 movements per minute, at least about 37,000 movements per minute, at least about 38,000 movements per minute, at least about 39,000 movements per minute, at least about 40,000 movements per minute, at least about 41,000 movements per minute, at least about 42,000 movements per minute, at least about 43,000 movements per minute, at least about 44,000 movements per minute, at least about 45,000 movements per minute, at least about 46,000 movements per minute, at least about 47,000 movements per minute, at least about 48,000 movements per minute, at least about 49,000 movements per minute, at least about 50,000 movements per minute, or any other frequency.

In an embodiment, the body portion 200 of an ultrasonic tongue scraper comprises an ultrasound generator configured to generate ultrasound. In some embodiments, the generated ultrasound is configured to be transmitted from the body portion 200 to and through the tongue scraper head portion 100. In certain embodiments, the generated ultrasound is transmitted to and through the tongue scraper head portion 100 via an elongated end 215 of the body portion that is placed in the interior of the tongue scraper head portion 100. In some embodiments, the frequency of the generated ultrasound can be controlled by a user via an ultrasound frequency selection button 210 or other input on the body portion 200. In other embodiments, the frequency of the generated ultrasound cannot be controlled by a user. In some embodiments, the body portion 200 comprises a light 205, display, or other means configured to indicate to a user whether ultrasound is currently being generated or not.

In some embodiments, the ultrasound generator is configured to generate ultrasound at a safe low-power medical frequency. For example, the ultrasound generator can be configured to generate ultrasound at a frequency of about 1.6 MHz or 1,600,000 Hertz which is equivalent to 96,000,000 pulses per minute. In certain embodiments, the ultrasound generator can be configured to generate ultrasound at a frequency of at least about 0 MHz, at least about 0.1 MHz, at least about 0.2 MHz, at least about 0.3 MHz, at least about 0.4 MHz, at least about 0.5 MHz, at least about 0.6 MHz, at least about 0.7 MHz, at least about 0.8 MHz, at least about 0.9 MHz, at least about 1.0 MHz, at least about 1.1 MHz, at least about 1.2 MHz, at least about 1.3 MHz, at least about 1.4 MHz, at least about 1.5 MHz, at least about 1.6. MHz, at least about 1.7 MHz, at least about 1.8 MHz, at least about 1.9 MHz, at least about 2.0 MHz, at least about 2.1 MHz, at least about 2.2 MHz, at least about 2.3 MHz, at least about 2.4 MHz, at least about 2.5 MHz, at least about 2.6 MHz, at least about 2.7 MHz, at least about 2.8 MHz, at least about 2.9 MHz, at least about 3.0 MHz, or any other frequency.

Method(s) of Use

In certain embodiments, the ultrasonic tongue scraper can be manually moved in a longitudinal, diagonal, horizontal or circular direction along the surface of a tongue to scrape bacteria, including bacterial biofilm, and/or other materials. In some embodiments, a leading edge 105 of the outer portion 115 is configured to do substantially most of the scraping as the ultrasonic tongue scraper is moved in a longitudinal direction along the surface of a tongue.

In some embodiments, mechanical vibration is generated by the ultrasonic tongue scraper to assist the manual scraping. In certain embodiments, the ultrasonic tongue scraper can generate mechanical vibration in one or more directions. For example, the ultrasonic tongue scraper can generate mechanical vibration along the longitudinal axis of the surface of a tongue and/or along an axis that is diagonal or perpendicular to the longitudinal axis. With the aid of mechanical vibration(s), the area and force of contact between the ultrasonic tongue scraper and the surface of a tongue can be increased to provide more efficient and/or effective cleaning.

In certain embodiments, ultrasound is generated by the ultrasonic tongue scraper to assist the manual scraping and/or mechanical vibration. For example, in some embodiments, the ultrasonic tongue scraper can generate and transmit ultrasound to break up bacterial chains, cell walls, and/or adhesions of bacteria and/or other debris on the surface of the tongue. In certain embodiments, the transmitted ultrasound can break up bacterial chains, cell walls, and/or adhesions of bacteria and/or other debris with or without actual contact to allow for more efficient cleaning with the same area and/or force of contact between the ultrasonic tongue scraper and the surface of a tongue.

In some embodiments, use of an embodiment of an ultrasonic tongue scraper as described herein reduces bacterial growth and/or other undesirable materials on the surface of a tongue by at least about 99%, at least about 98%, at least about 97%, at least about 96%, at least about 95%, at least about 94%, at least about 93%, at least about 92%, at least about 91%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, at least about 10%, at least about 5% or any other percentage.

Experiments

Studies are developed to show that an embodiment of an ultrasonic tongue scraper as described herein can be utilized to more effectively clean the surface of a tongue compared to manual tongue scrapers.

In an in vitro study, initial bacterial growth of bacteria placed on a fine-celled sponge-like material is measured. Bacteria on a fine-celled sponge-like material are scraped with a manual tongue scraper that is widely available (control). Bacteria on a fine-celled sponge-like material are scraped with an embodiment of an ultrasonic tongue scraper as described herein (variable). Bacterial growth and/or bacterial chain rupture in the resulting control and variable fine-celled sponge-like material after scraping are measured and compared to the corresponding initial values to determine a corresponding percentage decrease in bacterial growth and/or bacterial chain rupture after scraping. The percentage decrease in bacterial growth and/or increase in bacterial chain rupture after scraping with an ultrasonic tongue scraper in vitro is found to be unexpectedly higher than after scraping with a manual tongue scraper. In some studies, scraping bacteria in vitro with an embodiment of an ultrasonic tongue scraper as described herein is found to more effectively decrease bacterial growth and/or increase bacterial chain rupture than scraping with a manual tongue scraper by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, about 100%, or any other percentage.

In an in vivo study, initial bacterial growth of bacteria on the surface of a tongue(s) of a human or non-human animal is measured. Bacteria, including bacterial biofilm, located on some tongue surfaces are scraped with a manual tongue scraper that is widely available (control). Bacteria, including bacterial biofilm, located on other tongue surfaces are scraped with an embodiment of an ultrasonic tongue scraper as described herein (variable). Bacterial growth and/or the rupture of bacterial chains on control and variable tongue surfaces after scraping are measured and compared to the corresponding initial values to determine a corresponding percentage decrease in bacterial growth and/or the rupture of bacterial chains after scraping. The percentage decrease in bacterial growth and/or increase in the rupture of bacterial chains after scraping with an ultrasonic tongue scraper in vivo is found to be unexpectedly higher than after scraping with a manual tongue scraper. In some studies, scraping bacteria in vivo with an embodiment of an ultrasonic tongue scraper as described herein is found to more effectively decrease bacterial growth and/or increase in the rupture of bacterial chains than scraping with a manual tongue scraper by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, or any other percentage.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. The headings used herein are for the convenience of the reader only and are not meant to limit the scope of the inventions or claims.

Although the embodiments of the inventions have been disclosed in the context of a certain preferred embodiments and examples, it will be understood by those skilled in the art that the present inventions extend beyond the specifically disclosed embodiments to other alternative embodiments and/or uses of the inventions and obvious modifications and equivalents thereof. In addition, while a number of variations of the inventions have been shown and described in detail, other modifications, which are within the scope of the inventions, will be readily apparent to those of skill in the art based upon this disclosure. It is also contemplated that various combinations or subcombinations of the specific features and aspects of the embodiments may be made and still fall within one or more of the inventions. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above.

What is claimed is:

1. An ultrasonic tongue scraper for removing bacteria from a surface of a tongue, wherein the ultrasonic tongue scraper comprises:
a body portion, wherein the body portion comprises a power source and an ultrasound generator configured to generate ultrasound; and
a tongue scraper head portion, wherein the tongue scraper head portion is selectively removable from the body portion,
wherein the tongue scraper head portion comprises a leading edge that is shaped and configured to be used to contact the tongue and manually scrape bacteria off of the surface of the tongue, and transmit ultrasound generated by the ultrasound generator to further assist the manual scraping.

2. The ultrasonic tongue scraper of claim 1, wherein the transmitted ultrasound assists the manual scraping by breaking up chains of bacteria on the surface of the tongue.

3. The ultrasonic tongue scraper of claim 1, wherein the ultrasound generator is configured to generate a continuous or pulsating ultrasound wave at a frequency of about 1.6 MHz.

4. The ultrasonic tongue scraper of claim 1, further comprising a vibration frequency selection input configured to change a frequency of the mechanical vibration.

5. The ultrasonic tongue scraper of claim 4, wherein the frequency of the mechanical vibration is among one of about 18,000, about 9,000, and about 0 movements per minute.

6. The ultrasonic tongue scraper of claim 1, further comprising an ultrasound indicator configured to indicate whether ultrasound is being generated by the ultrasound generator.

7. The ultrasonic tongue scraper of claim 1, wherein the tongue scraper head portion is configured to be selectively replaced with a brush head portion.

8. The ultrasonic tongue scraper of claim 1, wherein the body portion further comprises a motor configured to generate mechanical vibration.

9. The ultrasonic tongue scraper of claim 8, wherein the leading edge is configured to transmit mechanical vibration generated by the motor to assist the manual scraping.

10. A method of scraping bacteria from a surface of a tongue, the method comprising:
attaching a tongue scraper head portion to a body portion, wherein the tongue scraper head portion is selectively removable from the body portion;
manually scraping bacteria off of the surface of the tongue by contacting the surface of the tongue with a leading edge of the tongue scraper head portion;
applying mechanical vibration to assist the manual scraping, wherein the mechanical vibration is generated by the body portion; and
applying ultrasound to assist the manual scraping, wherein the ultrasound is generated by the body portion.

11. The method of claim 10, further comprising breaking up chains of bacteria on the surface of the tongue by applying ultrasound.

12. The method of claim 10, wherein the generated ultrasound comprises a continuous or pulsating frequency of about 1.6 MHz.

13. The method of claim 10, further comprising selecting a frequency of the mechanical vibration.

14. The method of claim 13, wherein the frequency of the mechanical vibration is selected among one of about 18,000, about 9,000, and about 0 movements per minute.

15. The method of claim 10, wherein the ultrasonic tongue scraper indicates whether ultrasound is being generated.

16. The method of claim 10, further comprising detaching the tongue scraper head portion and attaching a brush head portion.
